(19) United States
(12) Patent Application Publication
(10) Pub. No.: US 2005/0118120 A1
(43) Pub. Date: Jun. 2, 2005

(54) Viscoelastic cosmetic composition for styling and conditioning of human hair

(76) Inventors: Marina Azizova, Rowayton, CT (US); David Budek, Stamford, CT (US); Rushi Tasker, Trumbull, CT (US)

Correspondence Address: MELVIN I. STOLTZ, ESQ., 51 CHERRY STREET, MILFORD, CT, 06460, US

(21) Appl. No.: 10/725,182

(22) Filed: Dec. 1, 2003

(51) Int. Cl. A61K 7/135; A61K 7/06; A61K 7/11

(52) U.S. Cl. 424/62; 424/70.15

(57) ABSTRACT

The sensory attributes of cosmetic compositions are very important to form positive consumer's perception of the product. The sensory attributes can vary significantly depending on the formulation employed. In the present invention, a particularly beneficial balance of attributes was attained to create a usable viscoelastric composition that can be poured as a liquid matter, handled as a solid matter and applied to hair as a styling and conditioning product. This composition comprises a unique blend of ingredients in an aqueous medium which achieves the desired results.

### FIELD OF THE INVENTION

This invention relates to aqueous hair styling composition than exhibits unusual viscoelastic behavior and can be used to condition and style hair.

### BACKGROUND OF THE INVENTION

Only a few materials and a small number of cosmetic formulations and food products can be described as Newtonian fluids, that is, their viscosities are not affected by shear. The viscosity of a Newtonian fluid is dependent on temperature but not on shear rate and time. All gases and most liquids with simpler molecular formula and low molecular weight such as water, benzene, ethyl alcohol, glycerine hexane and most solutions of simple molecules are Newtonian fluids. Some other examples of Newtonian fluids are: low viscosity face lotions, styling sprays, milk, and mineral oil.

The rest of the fluids are non-Newtonian as their viscosities increase or decrease as the applied shearing stress increases.

There are different classes of non-Newtonian fluids including: Bingham plastic, shear-thinning, and shear thickening.

Bingham-plastic products resist a small shear stress but flow easily under larger shear stresses. e.g. tooth-paste, jellies, and some slurries.

Shear thinning fluids (also called pseudoplastic) is the largest group of non-Newtonian fluids: viscosity decreases with increasing shear rate. Examples of shear thinning fluids are: creams, gels, shampoo and conditioners, slurries, fruit juice concentrates, sour crème, ketchup. Usually, it is desirable to create a shear-thinning cosmetic composition that can spread easily in the hands, on the skin or on hair.

Shear-thickening products (also called dilatant) are uncommon: viscosity increases with increasing shear rate. There are few examples of shear-thickening fluids, Silly Putty is one, suspensions of starch and sand also exhibit shear-thickening properties. It is known in the industry that certain associating polymers in selective solvents can exhibit shear thickening behavior over a limited range of concentration. It is generally caused by shear-induced changes in the structure of the material. However, the details of the mechanism of shear thickening in various systems are quite diverse and often poorly understood.

The determination of Newtonian fluid viscosity can be readily accomplished wit a single point measurement, regardless of the shear rate, since viscosity is independent of the shear rate. However, the flow and behavior of non-Newtonian fluid can only be described as a relationship of shear stress and shear rate.

Complex emulsions, particularly the ones containing gel network possess a quality known as elasticity, that further complicates their behavior.

Elasticity is a quality of the product to resist the deformation that is being applied to it. Ideal fluids, such as water, deform irreversibly, they flow and change their position when stress is applied. Ideal solids, such as a steel spring, deform elastically, they change their position, but the energy is stored. When the internal force is removed, they recoil to their original position. Viscoelastic materials exhibit both elastic and flow behavior and their viscosity changes as a function of shear rate.

Understanding complex emulsions came to the industry in the last twenty years with the development of instruments that can reliably apply a known stress or strain in small, discreet quantities. The first evaluations of elasticity were done using Creep test described by Thurston and Martin in 1978 (“Rheology of Pharmaceutical Systems: Oscillatory and Steady Shear of non-Newtonian Viscoelastic Liquids”, J. Pharm. Sci., 67(11):1499-1506).

### SUMMARY OF THE INVENTION

The present invention provides an unusual viscoelastic composition prepared from PPG-14 Palmeth-60 Hexyl Dicarbamate in combinations with Cocamidopropyl Betaine and water. This unique composition has been found to be highly effective in the conditioning and styling of hair.

Prior art products employ PPG-14 Palmeth-60 Hexyl Dicarbamate to create hairstyling compositions, where viscosity is obtained from association between the said compound and a surfactant. Suitable surfactants can be selected from the group of anionic, amphoteric, sugar surfactants and quaternary compounds. Such compositions can demonstrate Newtonian to pseudoplastic flow behavior. However, no teachings are found in the prior art which suggest the formulation of the present invention.

The structure of PPG-14 Palmeth-60 Hexyl Dicarbamate is as follows:
C_{16/18}—(PEG/PPG)_{60/14}-CO—NH—(CH₂)₆—NH—CO—(PEG/PPG)_{60/14}-C_{16/18}

Further the industry recommends using PPG-14 Palmeth-60 Hexyl Dicarbamate as an effective thickener for following applications:
(1) building low shear viscosity and Newtonian to pseudoplastic flow,
(2) providing synergistic thickening effect with other formulation ingredients,
(3) contributing to hair conditioning.

While the industry recommends using PPG-14 Palmeth-60 Hexyl Dicarbamate with surfactants to create Newtonian and shear-thinning composition, we have discovered that it can also create a unique viscoelastic gel composition when used with Cocamidopropyl Betaine. It is not completely clear how Cocamidopropyl Betaine affects the structure of PPG-14 Palmeth-60 Hexyl Dicarbamate, but the resulting product always exhibits an elastic behavior which is unusual for cosmetic compositions. The present invention demonstrates the following useful characteristics:
(1) can be poured out of a jar as a liquid (low viscosity at low shear)
(2) can be picked with a hand as a solid, rolled into ball that bounces off the wall (augmented viscosity and elasticity at high shear)
(3) can spread on hair creating a thin layer of product for effective styling and conditioning (reduced viscosity when shear is terminated)

In Table I, an overall, general formulation for the hair styling and conditioning composition of the present invention is provided. By referring to this formulation, the principal, required ingredients and quantity ranges for each ingredient are detailed, with each ingredient being detailed with its preferred percent by weight, based upon the weight of the entire composition.

TABLE I
Hair Styling and Conditioning Composition

| Ingredient | Weight % Range |
|---|---|
| PPG-14 Palmeth-60 Hexyl Dicarbamate | 1-10 |
| Cocamidopropyl Betaine | 3-30 |
| Water | q.s. to 100% |

In the preferred formulation of the commercial hair styling and conditioning composition of the present invention, one or more additional ingredients are incorporated into the base formulation defined in Table I. These additional ingredients include one or more selected from the group consisting of styling polymers, hair lightening compounds, perfumes, and preservatives. By referring to Table II, a preferred formulation for a commercial hair styling and conditioning composition of the present invention is fully detailed.

TABLE II
Hair Styling and Conditioning Composition

| Ingredient | Weight % Range |
|---|---|
| PPG-14 Palmeth-60 Hexyl Dicarbamate | 1-10 |
| Cocamidopropyl Betaine | 3-30 |
| Styling Polymers | 0-30 |
| Hair Lightening Compounds | 0-10 |
| Perfume | 0.01-1.0 |
| Preservative | 0.01-1.0 |
| Water | q.s. to 100% |

Finally, by referring to Table ll, the preferred detailed formulation for the hairstyling and conditioning composition of the present invention is provided. In this detailed formulation, each of the desired additives are provided, along with the preferred quantities employed for each ingredient. In each instance, the stated quantities represent the weight percent for each ingredient, based upon the weight of the entire composition.

TABLE III
Hair Styling and Conditioning Composition

| Ingredient | Weight % |
|---|---|
| PPG-14 Palmeth-60 Hexyl Dicarbamate | 5 |
| Cocamidopropyl Betaine | 15 |
| Vinylpyrrolidone Terpolymer | 3 |
| AMP - Acrylates/Allyl Methacrylate Copolymer | 10 |
| Preservative | 0.5 |
| Perfume | 0.2 |
| Water | q.s. to 100% |

In the preferred embodiment, the PPG-14 Palmeth-60 Hexyl Dicarbamate comprises Elfacos T212, which is manufactured by Akzo Nobel Surface Chemistry of Amersfoot, The Netherlands. Although this particular commercial product has been found to be effective in achieving the compositions of the present invention, any desired alternate, equivalent product can be employed with equal efficacy.

The invention accordingly comprises the several steps and the relation of one or more such steps with respect to each of the other, and the composition possessing the features, properties, and relation of ingredients which will be exemplified in the compositions hereinafter described, and the scope of the invention will be indicated in the claims.

### THE DRAWINGS

For a fuller understanding of the nature and objects of the invention, and the unique attributes of the compositions provided by the present invention, reference should be had to the following detailed description taken in connection with the accompanying drawings, in which:

FIG. 1, consisting of FIGS. 1A-1D, depict the different geometries employed by the equipment used to test the viscoelasticity of the present invention; and

FIG. 2 is a diagrammatic chart depicting the performance characteristics of the present invention as compared to prior art compositions.

### DETAILED DESCRIPTION

By employing the teaching of the present invention, a hair styling and conditioning gel composition is achieved which exhibits viscoelastic behavior which is virtually unknown for conventional prior art compositions. In accordance with the present invention, when the hair styling and conditioning gel is picked out of a jar, the composition resists squeezing in compression by becoming an instant solid. However, when the composition is slowly poured, the composition behaves as a liquid.

In order to demonstrate the unique attributes of the hair styling and conditioning gel composition of the present invention, the three formulations defined in Table IV were prepared and then tested in the manner detailed below. In addition, each of the samples were compared to two commercially available products, namely Joigel by Joico, a shear-thinning carbopol gel and mineral oil, a Newtonian fluid.

In order to test the elasticity of the hair styling and conditioning gel compositions of the present invention and compare the compositions of the present invention to the prior art products, a dynamic rheometer manufactured by TA Instruments was employed.

The dynamic rheometer is able to apply very small amounts of rotation or deformation in a dynamic or oscillatory fashion. This type of stress can be visualized as if the sample were being “vibrated” between parallel plates or concentric cylinders, as opposed to being sheared in a continuous fashion. The components of a modern dynamic rheometer enable this “vibratory” measurement to be applied to a sample in a controlled fashion while also controlling the sample temperature.

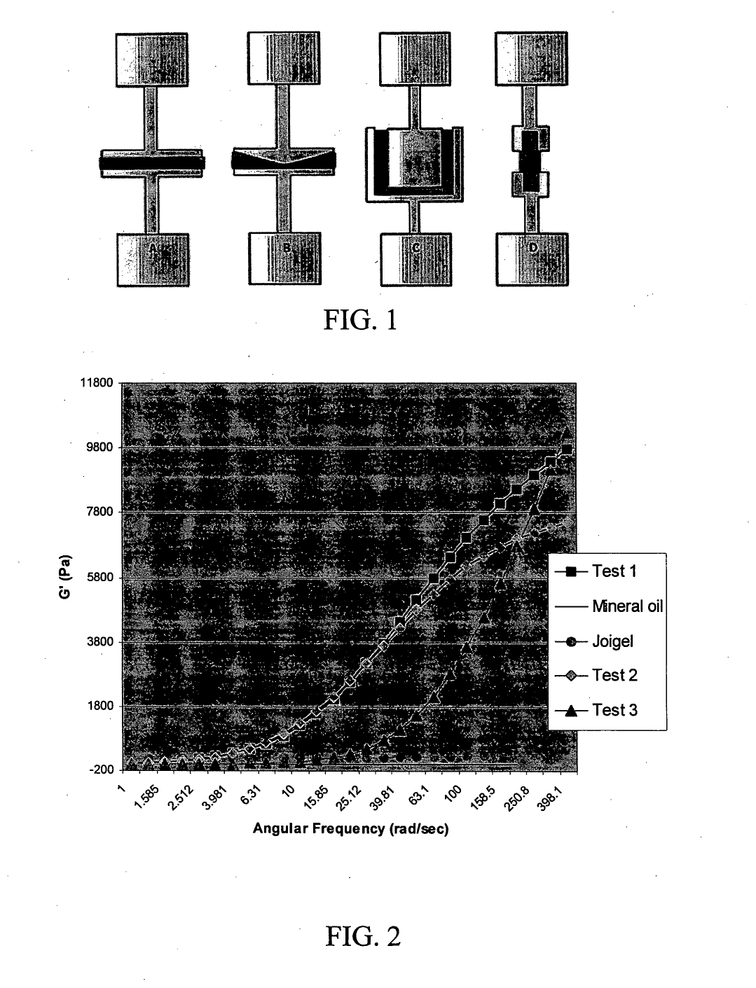

In FIG. 1, typical sample testing geometries for dynamic rheometers are depicted. As shown therein FIG. 1A depicts parallel plates, FIG. 1B shows a cone and a plate, FIG. 1C depicts concentric cylinders (couette), and FIG. 1D shows a solid or torsion rectangular. The appropriate geometry is dictated primarily by the properties of sample material, but may also be dictated by the desire to simulate a process or in situ application.

The significance of this dynamic testing method is that the resulting measurement is delivered in terms of discrete components of the material's viscosity or shear modulus, as opposed to the simple bulk viscosity reported by traditional viscometers.

When analyzed using a dynamic rheometer, the viscosity or shear modulus of a viscoelastic material may be resolved into components parts referred to as the “elastic” and “viscous” components:

The Complex Shear Modulus G* includes both viscous and elastic component.
G*=[(G′)2+(G″)2]½
where
G* is the dynamic shear modulus
G′ is the elastic or storage modulus, and
G″ is the viscous or loss modulus.

These component parts of the bulk viscosity or modulus have specific meaning in the context of the bulk properties of the material and are individually very sensitive to specific events occurring in the morphology or micro-structure, or even the nano-structure, of the material system. These same structural effects or phenomena are often invisible to traditional, steady-shear viscometry.

Further, the Elastic Module G′ of the gel viscosity represented by the ratio of stress over strain can be devised as follows:
G′=τ₀ cos δ/γ
The important functions of viscoelastic measurements are listed below:
Stress: τ=τ₀ cos ωt
Where τ₀₌shear stress at maximum amplitude
   ω=angular velocity
   t=time
Strain: γ=γ₀ cos (ωt−δ)
Where γ₀=shear strain at maximum amplitude
   δ=phase angle between shear stress and shear strain

A complete explanation of these functions is given by Deem, D. E. (1988) “Rheology of Dispersed Systems”, Marcel Dekker New York, pp 367-425.

As a detailed above, testing was conducted on all three formulations of the present invention, as well as on the two commercial products detailed above. In each test, the elastic modulus G′ was measured as a function of the shear stress (rad/second) applied to it. In FIG. 2, the results obtained from the tests conducted on each of the foregoing compositions are fully detailed, with the differences in the viscoelastic behavior of each product being fully provided.

As is evident from the results provided in FIG. 2, Joigel, a shear-thinning gel, shows no viscoelastic behavior, with its resulting elastic modulus (G′) remaining unchanged as shear stress increases. Similarly, mineral oil, a Newtonian fluid, does not change its elasticity due to shear. The elastic modulus (G′) of the mineral oil remains on the X-access, never deviating from zero in response to changes in stress.

In the hairstyling and conditioning gel compositions of the present invention, an increase in elasticity is produced with increasing shear stress. The results provided in FIG. 2 correlate with our observations that the product of the present invention becomes a solid when picked out of a jar.

In manufacturing the hair styling and conditioning gel of the present invention, it has been found that two separate vessels are employed with the compounds thereof the been separately prepared prior to being intermixed. In this regard, in the preferred manufacturing process, distilled water is place in a first vessel and the desired styling polymer is added into the water and mixed therein until completely dissolved. Then, a portion of the cocamidopropyl betaine is added to this vessel and the entire composition is heated to 65° C.

The remaining quantity of the cocamidopropyl betaine is added to a separate vessel and heated to 65° C. Then, the PPG-14 Palmeth-60 Hexyl Dicarbamate is added to this vessel and mixed therewith until homogeneous. Once the two phases in the two separate vessels are thoroughly mixed, the two phases are combined, and mixed for 10 minutes, or until uniform, maintaining the temperature at 65° C. throughout the mixing operation.

Thereafter, AMP—Acrylates/Allyl Methacrylate Copolymer is added to the composition, and mixed therein for 10 minutes, continuing to maintain the temperature at 65° C. Finally, the preservatives and fragrances are mixed therein until uniform. Any required distilled water is added, mixed for 10 minutes, with the temperature of the composition being maintained at 65° C. When the complete composition is poured into suitable containers while still at 65° C., and then allow to cool to room temperature.

By employing this preferred manufacturing process, three separate test compositions were prepared and tested as detailed above. The composition of each test formulation is detailed in Table IV, with the weight of each ingredient being provided, based upon the entire composition. The results of the test procedures are shown in FIG. 2.

TABLE IV

| | Example 1 | Example 2 | Example 3 |
|---|---|---|---|
| INGREDIENT | % by Wgt | % by Wgt | % by Wgt |
| PHASE A | | | |
| DI Water | 66.3000 | 90.3000 | 66.3000 |
| Vinylpyrrolidone Terpolymer | 3.0000 | 0.0000 | 0.0000 |
| Cocamidopropyl Betaine | 5.0000 | 2.0000 | 10.0000 |
| (30% active) | | | |
| PHASE B | | | |
| Cocamidopropyl Betaine | 10.0000 | 4.0000 | 20.0000 |
| (30% active) | | | |
| PPG-14 Palmeth-65 Hexyl | 5.0000 | 3.0000 | 3.0000 |
| Dicarbamate | | | |
| AMP-Acrylates/Allyl Methacrylate | 10.0000 | 0.0000 | 0.0000 |
| Copolymer | | | |
| Preservative | 0.5000 | 0.5000 | 0.5000 |
| Perfume | 0.2000 | 0.2000 | 0.2000 |
| DI Water | qs | qs | qs |

It will thus be seen that the objects set forth above, among those made apparent from the preceding description, are efficiently attained and, since certain changes may be made in carrying out the above process and in the compositions set forth without departing from the scope of the invention, it is intended that all matter contained in the above description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Particularly, it is to be understood that in the claims, ingredients or compounds recited in the singular are intended to include compatible mixtures of such ingredients wherever to sense permits.

1. A hair stylizing and conditioning gel possessing unusual viscoelastic properties, said gel comprising:
A. between about 1% and 100% by weight based upon the weight of the entire composition of PPG-14 Palmeth-60 Hexyl Dicarbamate;
B. between about 3% and 30% by weight based upon the weight of the entire composition of cocamidopropyl betaine; and
C. water forming the balance.

2. The hair styling and conditioning gel defined in claim 1, wherein said composition further comprises at least one selected from the group consisting of styling polymers, hair lightening compounds, fragrances, and preservatives.

3. The hairstyling and conditioning gel defined in claim 1, wherein said composition further comprises:
D. between about 0% and 30% by weight based upon the weight of the entire composition of at least one styling polymer;
E. between about 0% and 100% by weight based upon the weight of the entire composition of a hair lightening compound;
F. between about 0.01% and 1% by weight based upon the weight of the entire composition of a fragrance; and
G. between about 0.01% and 1% by weight based upon the weight of the entire composition of a preservative.

4. The hair styling and conditioning gel defined in claim 3, wherein the hairstyling polymer comprises at least one selected from the group consisting of vinylpyrrolidone polymers and acrylate copolymers.

5. The hair styling and conditioning gel defined in claim 4, wherein the acrylate copolymers are further defined as comprising AMP-Acrylates and Allyl methacrylate copolymers.

6. The hairstyling and conditioning gel defined in claim 4, wherein said vinlylpyrrolidone polymer is further defined as comprising vinylpyrrolidone terpolymer.

7. The hair styling and conditioning gel defined in claim 3, wherein said hair lightening agent is further defined as comprising hydrogen peroxide.

8. A method for manufacturing a hair styling and conditioning gel possessing unusual viscoelastic properties said method comprising:
A. adding a quantity of distilled water into a first vessel;
B. mixing a quantity of cocamidopropyl betaine into the water in the first vessel and heating the ingredients to between about 62° C. and 68° C., the quantity of cocamidopropyl betaine employed comprising between about 25% and 60% of the total quantity of this ingredient employed in the entire composition;
C. adding the remaining quantity of cocamidopropyl betaine to a second vessel and heating the second vessel to between about 62° C. and 68° C.;
D. adding PPG-14 Palmeth-60 Hexyl Dicarbamate into the second vessel and mixing the ingredients therein until homogeneous;
E. thoroughly intermixing the contents of each of vessel separately and, thereafter, combining the contents of the two vessels into a single vessel and mixing until uniform, while maintaining the temperature of the intermixed ingredients to between about 62° C. and 68° C.;
F. pouring the thoroughly mixed hairstyling and conditioning gel into suitable containers while the gel remains at a temperature ranging between about 62° C. and 68° C.; and
G. allowing the gel to cool to room temperature.

9. The method defined in claim 8, wherein the total quantity of cocamidopropyl betaine employed is further defined as comprising between about 3% and 30% by weight based upon the weight of the entire composition and the total quantity of PPG-14 Palmeth-60 Hexyl Dicarbamate is further defined as comprising between about 10% and 100% by weight based on the weight of the entire composition.

10. The method defined in claim 9, and comprising the additional step of mixing a hair styling polymer into the water contained in the first vessel and allowing the hairstyling polymer to be completely dissolved before adding the cocamidopropyl betaine thereto.

11. The method defined in claim 9, comprising the additional steps of
H. adding AMP-Acrylates/Allyl Methacrylate Copolymer to the composition after the thorough mixing of the components of the two vessels and continuing mixing the entire composition for about 10 minutes at an elevated temperature of between about 62° C. and 68° C.; and
I. thereafter intermixing preservatives and fragrances into the composition and continuing mixing the entire composition for about 10 minutes at an elevated temperature of between about 62° C. and 68° C.
